(19) United States
(12) Patent Application Publication
(10) Pub. No.: US 2015/0057726 A1
(43) Pub. Date: Feb. 26, 2015

(54) Adjustable Cold Packs for Hands and Feet

(71) Applicant: Stephanie B. Plass, Clifton, NJ (US)

(72) Inventor: Stephanie B. Plass, Clifton, NJ (US)

(21) Appl. No.: 14/184,761

(22) Filed: Feb. 20, 2014

Related U.S. Application Data
(60) Provisional application No. 61/868,686, filed on Aug. 22, 2013.

(51) Int. Cl. A61F 7/10

(52) U.S. Cl. CPC A61F 7/10; A61F 2007/0036
USPC 607/111; 607/112

(57) ABSTRACT

Provided is a plurality of reusable freezable cold packs that are applied to a user's extremity via a strap assembly. The device includes a strap assembly that resembles an adjustable bottomless sandal or a wrist brace. The cold packs include multiple attachment points for use with the strap assembly. The strap assembly comprises an elastic strap and a fastener to enable a user to apply the cold packs to a desired area on hands and feet without holding it in place for a prolonged period of time. The freezable cold packs help sooth pain on contact and eliminate the need to use hard ice cubes, which may cause pain or melt and cause water to drip on the floor, furniture, and other surfaces. The present device provides a simple and convenient means for a user to apply cold therapy to his or her hands or feet.

### CROSS REFERENCE TO RELATED APPLICATION

This application claims the benefit of U.S. Provisional Application No. 61/868,686 filed on Aug. 22, 2013, entitled “Hand and Toe Packs.” The above identified patent application is herein incorporated by reference in its entirety to provide continuity of disclosure.

### BACKGROUND OF THE INVENTION

1. Field of the Invention

The present invention relates to cold packs for upper and lower extremities. More specifically, the present invention describes freezable cold packs and a strap assembly adapted for securing around a user's hands and feet. The present invention resembles a wrist brace or a bottomless sandal when it is assembled. The cold packs are secured in place via hook and loop fasteners, snap fasteners, or the like. To use the device, the cold packs are chilled in a freezer. When the cold packs are chilled to a desired temperature, a user positions the cold packs directly over a desired area on hands and feet. The strap assembly is used to secure the cold packs in place, which allows the user to resume most normal activities without holding a conventional cold pack for an extended period of time.

Many injuries need to be treated with cold packs right away to help prevent further injury. Icing may help relieve pain, prevent stiffness, and reduce inflammation to the injured area. Wrapping an ice pack around hands and feet can be difficult, and holding the ice pack in place can be inconvenient. Individuals typically fill a plastic bag with ice and tape the bag to the injured site. Unfortunately, hard ice cubes and bulky ice pack can place undue stress and pressure on the injury, causing further pain. Holding an ice pack in place by hand can also make the hand numb due to prolonged exposure to cold temperature. Melted ice can also leak and create make a mess on various surfaces. Due to such inconveniences, some individuals may skip the icing process altogether, which may prolong the healing process.

The present invention relates to a new and improved cold pack designed for use on hands and feet. The cold pack of the present invention comprises a refrigerant gel or liquid made of non-toxic materials that remain a slow flowing gel. Additives to improve the properties of gel or liquid may be used. For example, substances can be added to prevent bacterial growth in the pack, or to prevent the liquid from solidifying so it remains a thick gel throughout use. The device includes a set of differently shaped cold packs and a strap assembly that resembles a wrist brace or a bottomless sandal. The cold packs help soothe pain on contact and eliminate the use of hard ice cubes and any further pain associated with their use. The cold packs can be manipulated to accommodate different body contours and shapes, providing comfort to the user. The cold packs include multiple attachment points for use with the straps, which securely position the cold packs to a desired area on the hand or the foot. The straps preferably include a hook and loop fastener, which enables a user to adjust the straps to accommodate individuals of all ages and sizes. The present invention provides a convenient and safe way to apply cold therapy to hands and feet by allowing a user to secure the cold packs in place a hands-free manner. Furthermore, the cold packs of the present invention prevent melted ice from dripping on the floor, furniture, and other surfaces.

2. Description of the Prior Art

Devices have been disclosed in the prior art that relate to therapeutic cold packs. These include devices that have been patented and published in patent application publications. Some of these patents describe a footwear with a removable insole. Other patents disclose a footpad that includes built-in ice packs. These devices, however, do not disclose ice packs with an adjustable strap assembly that provides versatility and comfort to a user. The prior art also fail to disclose ice packs adapted for various parts of hands and feet. The following is a list of devices deemed most relevant to the present disclosure, which are herein described for the purposes of highlighting and differentiating the unique aspects of the present invention, and further highlighting the drawbacks existing in the prior art.

These prior art devices have several known drawbacks. For example, U.S. Pat. No. 5,961,544 to Goldman describes a convertible therapeutic sport sandal. The Goldman device includes a removable sole filled with a solution formulated to retain hot or cold temperatures for a period of time. The insole is adapted to attach to a footwear. While the Goldman device provides cold treatment for the bottom of a foot, it does not address the issue of icing other parts of a foot, such as toes or ankles. The present invention addresses these issues by providing cold packs adapted to fit around different parts of feet. Specifically, the cold packs of the present invention are shaped to wrap around individual toes, feet, and ankles. Additionally, the cold packs can be used to ice a user's hand. In contrast, the footwear with a removable sole cannot be secured to the user's hand.

Similarly, U.S. Pat. No. 8,475,511 to Snyder describes a device for applying cold therapy to feet. The Snyder device comprises a foot pad that is formed of an ice pack, further comprising a container or shell for receiving and containing a substance capable of maintaining low temperature for an extended period of time. Similar to the Goldman device, the Snyder device also provides no means to apply cold therapy to upper parts of feet, as well as toes and ankles. Additionally, the Snyder foot pad comprises a premolded shell, comprising a heel cup and a raised and contoured arch portion. The premolded shell disclosed in Snyder further limits its use to feet, and the premolded shells cannot be adjusted to an individual user's foot. In this regard, the cold packs of the present invention provide versatility to the user because the cold packs may be shaped to fit around toes, feet, and ankles, as well as fingers, hands, and wrists. Furthermore, because the cold packs are malleable casings, the packs can be manipulated into the shape of different parts of the user's foot or hand.

Another device, U.S. Pat. No. 5,409,500 to Dyrek describes a therapeutic cold pack. The cold pack has a plurality of cooling gel compartments and straps for temporary affixation to an area of a human body. While the therapeutic cold pack disclosed by Dyrek is adapted for providing cold therapy to various parts of a human body, the Dyrek device does not disclose multiple attachment points adapted for an adjustable strap assembly. Rather, the Dyrek device comprises a fastener placed directly on a back surface of the cold pack. Having a fastener directly on the surface of the cold pack can be problematic, however. For instance, the condensation from the cold pack may cause the epoxy on back of the fastener to flake off after several uses. Alternatively, a barrier layer of the cold pack may rip if the fastener is snagged during use, causing the refrigerant gel contained within the compartments to leak or spill. The present invention addresses these issues by allowing a user to thread the straps through multiple attachment points and avoiding use of epoxy or the like. Furthermore, the Dyrek device does not disclose a cold pack shaped to fit around digits on hands or feet. The cold packs of the present invention can fit around fingers or toes of the user, allowing the user to provide cold therapy to broken fingers and toes.

Yet another prior art device, U.S. Pat. No. 5,129,391 to Brodsky also describes thermal packs adapted for various parts of a human body. The thermal packs comprise packages filled with freezable gel shaped for various parts of the body. Although the thermal packs disclosed in Brodsky may provide cold therapy to upper and lower extremities, the Brodsky device has limited use in that it cannot wrap around multiple digits at the same time. Additionally, the thermal packs for the digits are bulky and require a user to fold it over the top of the digits, limiting mobility and providing discomfort to the user. Thus, the Brodsky device does not provide a convenient way to apply ice to the toes and fingers. In contrast, the present invention includes a cold pack adapted for digits, comprising multiple digit openings for a user to slide the digits therethrough. The digit openings allow the user to utilize the tips of fingers or toes, providing convenience to the user to resume normal activities such as typing, gripping, and pressing.

The present invention discloses a set of cold packs with a strap assembly attached thereto. The cold packs of the present invention are adapted to fit around various parts of hands and feet. The cold packs further include several attachment points for the straps that facilitate in securing the cold packs in place. The present invention provides and effective and convenient way to apply cold therapy to hands and feet. While the devices disclosed in the prior art include various ice packs, none of these devices disclose a set of cold packs adapted to fit around various parts of both upper and lower extremities. It is therefore submitted that the present invention substantially diverges in design elements from the prior art and consequently it is clear that there is a need in the art for an improvement to existing cold pack devices. In this regard the instant invention substantially fulfills these needs.

### SUMMARY OF THE INVENTION

In view of the foregoing disadvantages inherent in the known types of cold pack devices now present in the prior art, the present invention provides a new improvement to cold packs wherein the same can be utilized for providing a convenient and safe way to apply the cold packs to a desired area on the hand or foot.

It is therefore an object of the present invention to provide a new and improved cold pack device that has all of the advantages of the prior art and none of the disadvantages.

It is another object of the present invention to provide an cold pack device that include multiple attachment adapted for use with an adjustable strap assembly to secure the cold packs in place on hands or feet of a user.

Another object of the present invention is to provide an cold pack device with a reusable freezable cold pack that helps soothe pain on contact and eliminates the use of hard ice cubes and any further pain associated with their use.

Yet another object of the present invention is to provide an cold pack device shaped to fit around various parts of hands and feet and accommodate different body contours and shapes of an individual user.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a view of the components for toes, feet, and ankles separately.

FIG. 2 shows a view of the components for fingers, hands, wrists and arms separately.

FIG. 3 shows a close up cross-sectional view of the toe or finger cold pack.

FIG. 4 shows a view of the cold packs and strap assembly in place for toes, feet, and ankles.

FIG. 5 shows a view of the cold packs and strap assembly in place for fingers, hands, wrists, and arms.

### DETAILED DESCRIPTION OF THE INVENTION

Reference is made herein to the attached drawings. Like reference numerals are used throughout the drawings to depict like or similar elements of the cold pack. For the purposes of presenting a brief and clear description of the present invention, the preferred embodiment will be discussed as used for a cold pack in place for hands and feet. The figures are intended for representative purposes only and should not be considered to be limiting in any respect.

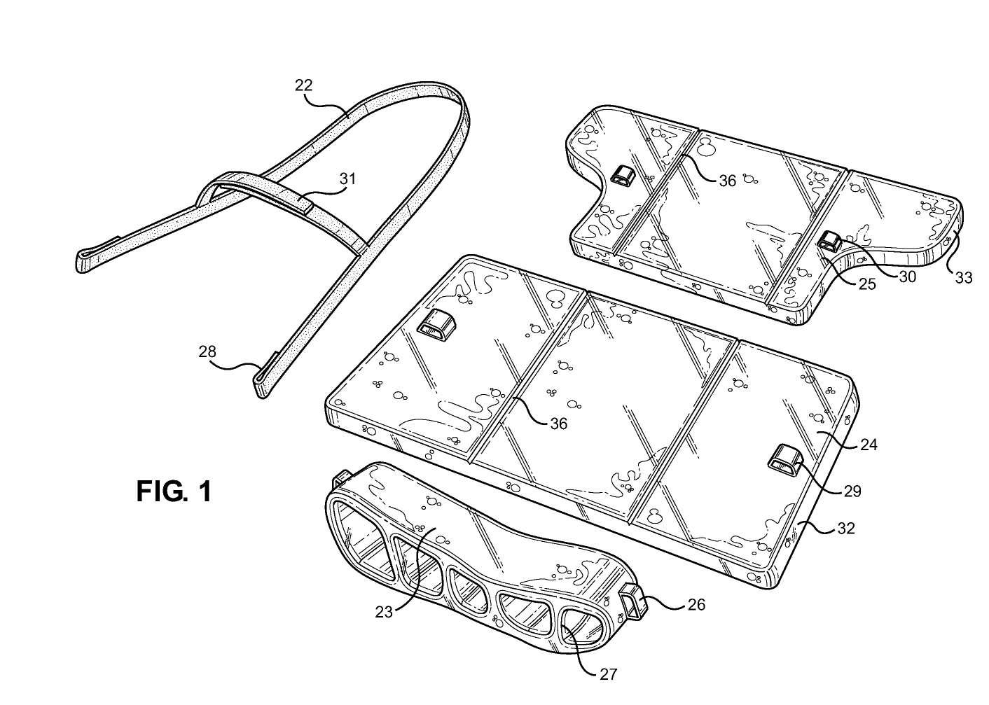

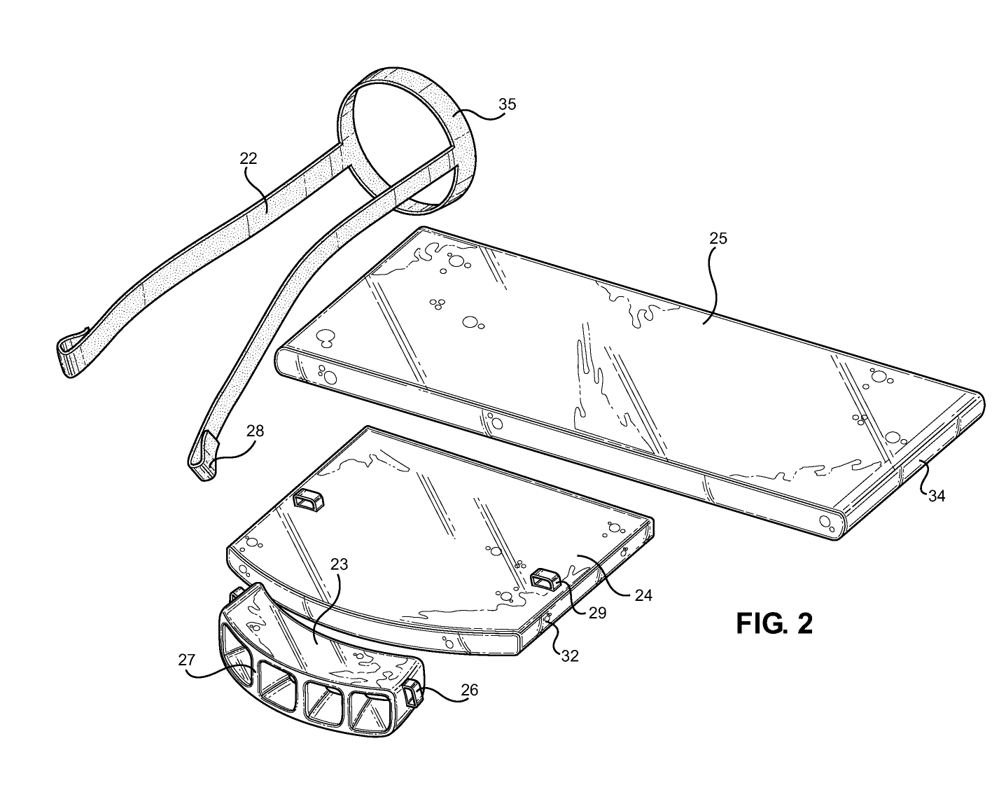

Referring now to FIGS. 1 and 2, there are shown views of the separate components of the present invention. The device preferably comprises a strap system and a plurality of cold packs. In an exemplary embodiment for lower extremities as shown in FIG. 1, the device encompasses a first cold pack 23 adapted to be secured around a user's toes, a second cold pack 24 adapted to be secured around a user's feet, and a third cold pack 25 adapted to be secured around a user's ankles. In an alternative exemplary embodiment for upper extremities as shown in FIG. 2, the device encompasses a first cold pack 23 adapted to be secured around a user's fingers, a second cold pack 24 adapted to be secured around a user's hand, and a third cold pack 25 adapted to be secured around a user's wrist. The cold packs 23, 24, 25 further comprise a flexible casing that is filled with freezable liquid material. The casing may comprise durable plastic or another suitable material. The casing is completely sealed around the edges to prevent the freezable material contained within its interior volume from leaking. The exterior surface of the cold packs 23, 24, 25 is malleable, but substantially firm, and can be compressed against the user's features and molded thereto when deployed. This allows the cold packs to contour around various parts of lower and upper extremities, providing comfort to a user and direct contact between the frozen or substantially frozen cold pack.

The freezable material contained in the casing is preferably water or a water-based gel material that is capable of obtaining a frozen or substantially frozen state when placed in a conventional freezer. When substantially frozen, the material will remain a slow flowing gel that will not spill easily or cause contamination if the casing breaks. The freezable material may comprise a family of gel materials, including hydroxyethyl cellulose or vinyl-coated silica gel. The non-toxic gel can absorb a considerable amount of heat due to the high enthalpy of fusion of water. Accordingly, the freezable gel material is suitable for alleviating the pain from minor injuries.

The cold pack 23 is adapted for use with digits of upper as well as lower extremities. The cold pack 23 has a substantial cuboid shape and comprises a plurality of openings for a user to insert his or her digits therethrough. A plurality of dividers 27 between the openings allow a user to insert a single digit in each opening of the cold pack 23. The cold pack 24 is substantially rectangular and is adapted to cover the bottom or upper portion of a hand or a foot. The lateral sides 32 of the cold pack 24 extend up the sides of the hand or a foot. The cold pack 24 also includes at least one divider wall 36 disposed within its interior volume to separate its interior volume, which facilitate bending of the cold pack 24 when in use to conform to a user's a foot or palm of a hand. The cold pack 25 is adapted to wraparound either a wrist or an ankle.

In an exemplary embodiment for lower extremities, the cold pack 25 is adapted to be secured around a user's ankle. It is a modified-rectangular shape having curved lateral portions 33 adapted to conform to a user's ankle and foot upper substantially. The cold pack 25 also includes at least one divider wall 36 disposed within its interior volume to facilitate bending the cold pack 25 around a user's ankle and foot upper. In an exemplary embodiment for upper extremities, the cold pack 25 is adapted to be secured around a user's wrist. The cold pack 25 adapted for use with a user's forearm is substantially rectangular in shape and it has lateral edges 24. In either embodiment, the cold pack 25 partially extends towards a forearm or a lower leg.

The cold packs 23, 24, 25 are held in place using an adjustable strap 22 comprising preferably elastic material. In one embodiment of the fastener shown in FIG. 1, the adjustable strap 22 includes a U-shaped end disposed opposite of two ends adapted for looping through multiple attachment points 26, 29, 30. A fastener 28 is disposed at each end enable a user to thread the two ends through the multiple attachment point 26 and fold it onto itself and form a closed loop after being inserted into the attachment point 26. The fastener 28 may preferably comprise a hook and loop fastener, a snap fastener, or the like. The strap 22 further includes a cross strap 31 that crosses the user's foot laterally while the U-shaped end of the strap 22 wraps over the cold pack 25 around the back of the user's ankle. The cross strap 31 also comprises a fastener to allow the user to adjust the strap 22 tightly around the cold pack 23. Additionally, unfastening the cross strap 31 of the strap 22 allows the user to easily slip his or her foot in and out of the cold packs 23, 24, 25. The multiple attachment points 26, 29, 30 are a U-shaped protrusion adapted to accept the two strap ends therethrough. When in use, the two ends of the adjustable strap 22 may be connected to multiple attachment points 26 on either side of the cold pack 23 adapted for a user's toes. The straps 22 are then threaded through the multiple attachment point 29 on cold pack 24 adapted for a user's feet and multiple attachment 30 on cold pack 25 adapted for a user's ankles.

In another embodiment of the fastener as shown in FIG. 2, the strap 22 also includes two ends adapted for fastening the cold packs 23, 24, 25 as discussed above. More specifically, the strap 22 threads through multiple attachment points 26 on the cold pack 23 adapted for fingers, and multiple attachment point 29 on the cold pack 24 adapted for hands. One end of the strap 22 further includes a loop 35 adapted for the user to slide his or her forearm therethrough. The looped end 35 of the strap 22 enables the user to keep the cold pack 25 wrapped around the user's wrist or forearm area. As such, a third set of multiple attachment point on the cold pack 25 adapted for wrists may or may not be included. The adjustable strap 22 provides a hands-free method to hold the cold packs 23, 24, 25 in place on a user's fingers, hands, and wrists, respectively. The elastic material also accommodates users of various sizes and ages to use the present invention.

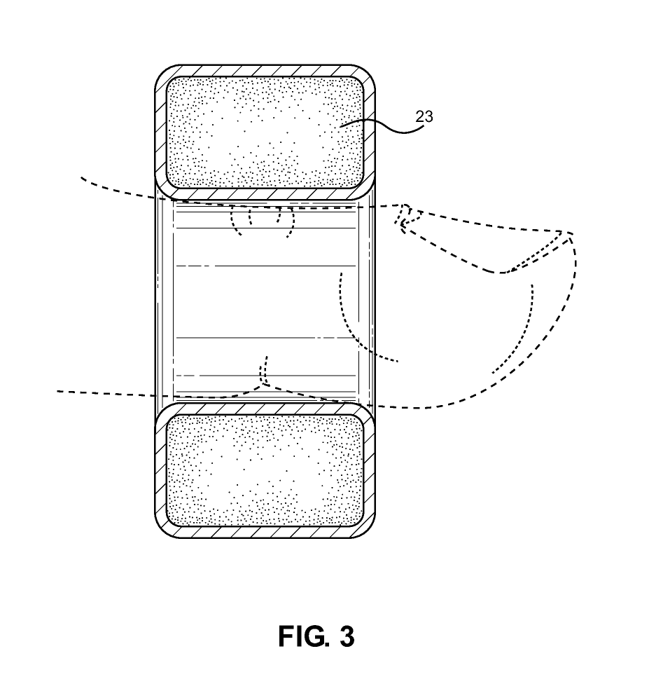

Referring now to FIG. 3, there is shown a close up cross-sectional view of the cold pack 23 adapted for fingers and toes. Each finger or toe is slid through a separate opening on the cold pack 23. The cold pack 23 further comprises a plurality of dividers to keep the fingers and toes separate. The dividers comprise the same flexible casing that is filled with freezable liquid material as used for the cold pack 23. As such, the cold pack 23 enables a user to provide cold therapy to the top, bottom, and sides of the digits. The cold pack 23 can be positioned such that it covers tips of the digits or the lower parts of the digits toward the knuckles. The cold pack 23 provides cushioning and holds the injured digits in place while providing cold therapy.

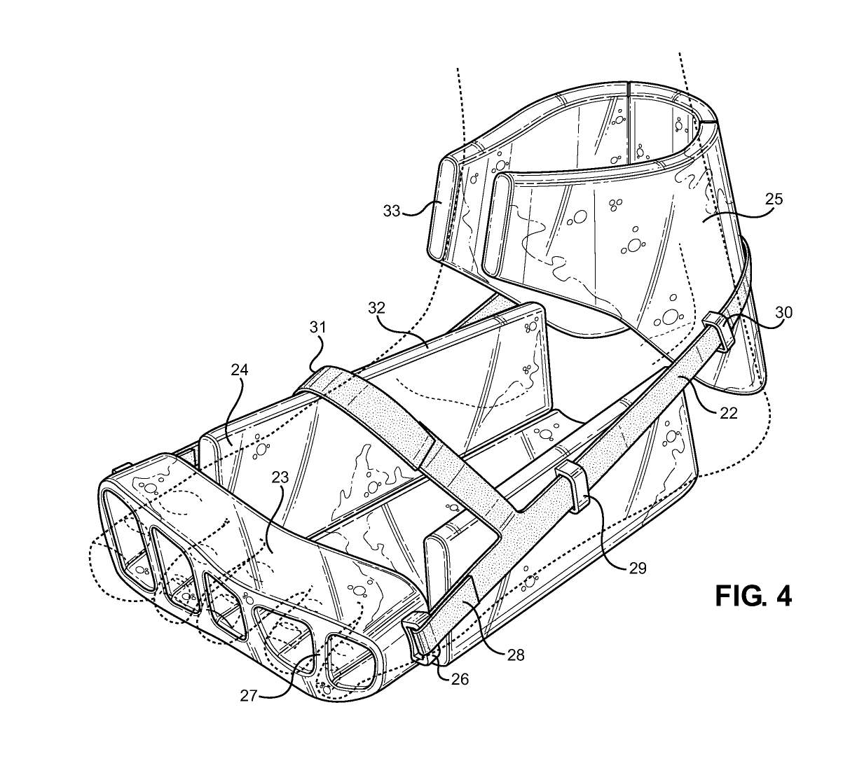

Referring now to FIG. 4, there is shown a view of the cold packs 23, 24, 25, and the adjustable strap 22 for lower extremities in use as worn by a user. The present invention includes a set of cold packs 23, 24, 25, adapted for toes, feet, and ankles, respectively. The cold packs further include an adjustable strap 22 that resembles a footwear when assembled. The cold packs 23, 24, 25 include multiple attachment points 26, 29, 30, respectively. When used with the adjustable strap 22, the multiple attachment points 26, 29, 30 provide a hands-free method to secure cold packs onto a user's lower extremity.

The first cold pack 23 resembles a toe separator and includes a plurality of dividers 27 for insertion between the toes or fingers. An attachment point 26 is positioned on either side of the first cold pack 23 to which a set of straps 22 may be attached thereto. The strap 22 secures the cold pack 23 in place by extending along the sides of a user's foot toward the back of the user's ankle. The second cold pack 24 has a substantially rectangular shape and can be wrapped under the arch of the foot or top of the foot. When the cold pack 24 is positioned over the top or bottom of the foot, the cold pack 24 is adapted to follow the contour of the foot. The cold pack 24 includes at least one divider wall 36 disposed within its interior volume to facilitate bending of the cold pack 24 when in use to conform to a user's a foot. When the cold pack 24 is bent, the sides 32 of the cold pack 24 extend partially up the sides of the user's foot to provide more coverage. This also allows the adjustable strap 22 to be placed over the sides 32 of the cold pack 24 so that the strap 22 can thread through additional attachment points 29 placed on either lateral side 32 of the cold pack 24.

The third cold pack 25 is adapted to be positioned around the backside of the user's ankle and anklebone. The cold pack 25 is a modified-rectangular shape with curved lateral portions 33 adapted to wrap over the user's ankle bone. The cold pack 25 also includes at least one divider wall disposed within its interior volume near the curved lateral portions 33 to separate said interior volume and facilitate bending of the cold pack 25 when in use to conform to a user's ankle. After being positioned in place, the adjustable strap 22 is wrapped around the cold pack 25 on the backside of the ankle. Additionally, the straps 22 may thread through multiple attachment points 30 on the rounded protruding ends 33 of the cold pack 25. This allows a user to hold the cold packs without manually holding it for an extended period of time. The cold packs 23, 24, and 25 can be used together or individually. After use, they can be returned to the freezer for additional uses.

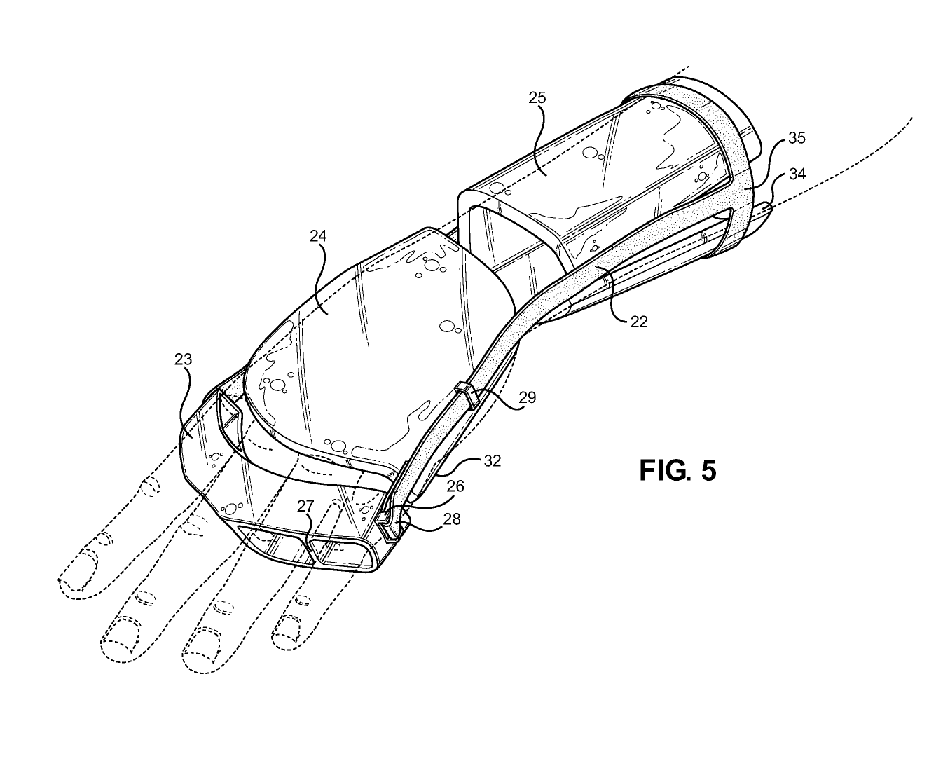

Referring now to FIG. 5, there is shown a view of the cold packs 23, 24, 25 and the adjustable strap 22 as used for the upper extremities. When applied to the upper extremities, the cold packs 23, 24, 25, and the strap 22 resemble a wrist brace, allowing a user to provide cold therapy to fingers, hands, wrist, and forearms. The cold pack 23 that accepts the toes, also accepts the fingers. An attachment point 26 is located on either side of the cold pack 23 for use with the adjustable strap 22. The strap 22 is secured to each attachment point 26 on cold pack 23 and is extended along the sides of the user's hand from the fingers to the user's forearm. The cold pack 24 is substantially rectangular in shape, and it can be positioned over the palm of a user's hand or on top of the user's hand. The sides 32 of the cold pack 24 extend up the sides of the hand to provide more coverage to the user's hand. An attachment point 29 is also positioned near either lateral side edges 32 of the cold pack 24 to allow the user to position the strap 22 closer to the user's forearm. The cold pack 25 adapted for use with a user's wrist or forearm is also substantially rectangular in shape, but preferably larger than the cold pack 24. The cold pack 25 wraps around the user's wrist and forearm such that the sides 34 of the cold pack 25 nearly touch. The looped end 35 of the strap 22 encircles the cold pack 25 and holds the cold pack 25 in place. As disclosed above, the cold packs 23, 24, 25 can be used separately or together. In use, the present invention allows the user to resume most normal activities.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A reusable cold pack adapted for upper or lower extremities, comprising:
an adjustable strap assembly having a first end and a second end, wherein at least one end comprises a fastener;
at least one freezable cold pack comprising a flexible casing having a sealed interior volume, an exterior surface, and a freezable liquid-like material within said interior volume capable of maintaining a low temperature for an extended period of time when cooled, frozen, or substantially frozen;
at least one of said freezable cold packs comprising a first and a second strap assembly attachment point disposed along opposite lateral portions thereof, said strap assembly capable of securing to said strap attachment points.

2) The reusable cold pack of claim 1, wherein said least one freezable cold pack further comprises:
a freezable cold pack comprising a substantial cuboid shape having a plurality of digit openings therethrough and a plurality of dividers between said digit openings adapted for insertion between a user's digits.

3) The reusable cold pack of claim 1, wherein said least one freezable cold pack further comprises:
a freezable cold pack substantially planar rectangular shape adapted to conform to a user's arch of a foot or palm of a hand.

4) The reusable cold pack of claim 3, wherein:
said freezable cold pack further comprises at least one divider wall disposed within said interior volume to separate said interior volume and facilitate bending of said second cold pack when in use to conform to a user's a foot or palm of a hand.

5) The reusable cold pack of claim 1, wherein said least one freezable cold pack further comprises:
a freezable cold pack having a modified-rectangular shape having curved lateral portions adapted to conform to a user's ankle and foot upper.

4) The reusable cold pack of claim 5, wherein:
said freezable cold pack further comprises at least one divider wall disposed within said interior volume to separate said interior volume and facilitate bending of said second cold pack when in use to conform to a user's ankle and foot upper.

6) The reusable cold pack of claim 1, wherein said least one freezable cold pack further comprises:
a first freezable cold pack comprising a substantial cuboid shape having a plurality of digit openings therethrough and a plurality of dividers between said digit openings adapted for insertion between a user's toes;
a second freezable cold pack substantially planar rectangular shape adapted to conform to a user's arch of a foot;
a third freezable cold pack having a modified-rectangular shape having curved lateral portions adapted to conform to a user's ankle.

7) The reusable cold pack of claim 6, wherein:
at least one divider wall disposed within said interior volume of said second freezable cold pack and said third freezable cold pack to separate said interior volume and facilitate bending of said second cold pack and said third cold pack when in use to conform to a user's a foot and a user's ankle.

8) The reusable cold pack of claim 1, wherein said least one freezable cold pack further comprises:
a first freezable cold pack comprising a substantial cuboid shape having a plurality of digit openings therethrough and a plurality of dividers between said digit openings adapted for insertion between a user's fingers;
a second freezable cold pack substantially planar rectangular shape adapted to conform to a user's hand or palm;
a third freezable cold pack having a rectangular shape adapted to conform to a user's wrist or forearm area.

9) The reusable cold pack of claim 8, wherein:
at least one divider wall disposed within said interior volume of said second freezable cold pack to separate said interior volume and facilitate bending of said second cold pack when in use to conform to a user's a hand.

10) The reusable cold pack of claim 1, wherein said fastener comprises hook and loop fastener material.

11) The reusable cold pack of claim 1, wherein said strap assembly further comprises a first and second lateral elastic strap attached to a looped end disposed opposite of said first end and said second end.

12) The reusable cold pack of claim 1, wherein said strap assembly further comprises:
an elongated lateral elastic strap forming a substantial U-shape having a first and second lateral strap portion between said first end and said second end;
a cross strap between said first and second lateral strap portions.

13) The reusable cold pack of claim 1, wherein said freezable liquid material further comprises a freezable, water-based gel material.

14) The reusable cold pack of claim 1, wherein said freezable liquid material further comprises water.

15) The reusable cold pack of claim 1, wherein said first and second strap assembly attachment point further comprises:
a U-shaped protrusion adapted to accept said first strap end and said second strap end therethrough;
said first strap end and said second strap further comprise a fastener comprising hook and loop fastener material adapted to allows said first strap end and said second strap to fold onto itself and form a closed loop after being inserted into said U-shaped protrusion.
